(19) United States
(12) Patent Application Publication
(10) Pub. No.: US 2014/0099262 A1
(43) Pub. Date: Apr. 10, 2014

(54) ISOTOPICALLY LABELED CDP-CHOLINE AND USES THEREOF

(71) Applicants: Rachel Katz-Brull, Modi'in-Maccabim-Re'ut (IL); Moshe Gomori, Jerusalem (IL)

(72) Inventors: Rachel Katz-Brull, Modi'in-Maccabim-Re'ut (IL); Moshe Gomori, Jerusalem (IL)

(73) Assignee: BRAIN WATCH LTD., Tel-Aviv (IL)

(21) Appl. No.: 14/123,343

(22) PCT Filed: May 31, 2012
(86) PCT No.: PCT/IL2012/050192

§ 371 Date: Dec. 2, 2013

Related U.S. Application Data
(60) Provisional application No. 61/492,080, filed on Jun. 1, 2011.

(51) Int. Cl. A61K 49/10; A61K 51/04; C07H 19/06

(52) U.S. Cl. CPC A61K 49/10; C07H 19/06; A61K 51/0491
USPC 424/1.73; 536/26.23; 424/9.35

(57) ABSTRACT

The present invention provides CDP-Choline (Citicoline) and any salt thereof comprising at least one isotopically labeled carbon atom directly bonded to at least one deuterium atom, a composition comprising said CDP-Choline, uses thereof, methods for diagnosing and evaluating a state, condition, or disease in a subject utilizing CDP-Choline of the invention and kits comprising CDP-Choline of the invention.

### FIELD OF THE INVENTION

This invention relates to isotopically labeled CDP-Choline, and uses thereof as a medical imaging agent in the diagnosis and evaluation of a condition, disease or disorder in a subject.

### BACKGROUND OF THE INVENTION

Cytidine diphosphate-choline (CDP-Choline) also known as Citicoline (INN), and cytidine 5′-diphosphocholine (5′-O-[hydroxy({hydroxy[2-(trimethylammonio)ethoxy]phosphoryl}oxy)phosphoryl]cytidine) is a naturally occurring, water soluble, compound that is used by the brain and body to make phospholipids, including phosphatidylcholine.

CDP-Choline is an endogenous intermediate in the generation of phosphatidylcholine from choline. Phosphatidylcholine comprises 30% of the grey matter of brain tissue. CDP-Choline is a psycho-stimulant (Giménez R, et al. 1991, British Journal of Pharmacology and Agut J, et al. 2006, Annals New York Academy of Sciences) and/or nootropic agent (Teather L A et al. 2005, Learning & Memory). Administration of CDP-Choline was shown to raise the levels of acetylcholine and improve brain metabolism and overall energy (Silveri M M 2008, NMR in Biomedicine).

CDP-Choline is sold in over 70 countries under a variety of brand names: Ceraxon and NeurAxon (Ferrer Therapeutics, NJ, USA), Cognizin (Kyowa Hakko Kogyo Co., Ltd, NY, USA), and Somazina (Ferrer International S.A., Barcelona, Spain). Somazina is indicated for the treatment of disturbance of consciousness resulting from head injuries, brain operation and acute stage of cerebral infarction. CDP-Choline is administered in doses of 500 mg or 1000 mg of the sodium equivalent. Somazina can be administered by intramuscular, slow intravenous route (3 to 5 minutes) or in deep perfusion and is compatible with all intravenous isotonic solutions. It can also be mixed with hypertonic glucosed serum. This brand is provided as tablets or ampules.

It is known in the art that CDP-Choline administration can act to reduce the extent of infarction, particularly cerebral infarction subsequent to cerebral ischemia (U.S. Pat. No. 5,872,108 and U.S. Pat. No. 5,827,832). It is also known in the art that CDP-Choline may act as a choline esterase inhibitor thereby increasing acetylcholine levels (WO199807431A1). It is known in the art that CDP-Choline may be considered a vasodilator (Pinardi G et al. 1994, General Pharmacology).

Magnetic resonance imaging and spectroscopy (MRI/MRS) has become an attractive diagnosing technique in the last three decades. Due to its non-invasive features and the fact that it does not involve the exposure of the diagnosed patient to potentially harmful ionizing radiation, MRI has become a leading diagnosing imaging procedure implemented in many fields of medicine.

The underlying principle of MRI and MRS is based on the interaction of atomic nuclei with an external magnetic field. Nuclei with spin quantum number I=½ (such as ¹H, ¹³C, and ¹⁵N) can be oriented in two possible directions: parallel (“spin up”) or anti-parallel (“spin down”) to the external magnetic field. The net magnetization per unit volume, and thus the available nuclear magnetic resonance (NMR) signal, is proportional to the population difference between the two states. If the two populations are equal, their magnetic moments cancel, resulting in zero macroscopic magnetization, and thus no NMR signal. However, under thermal equilibrium conditions, slightly higher energy is associated with the “spin down” direction, and the number of such spins will thus be slightly smaller than the number of spins in the “spin up” state.

An artificial, non-equilibrium distribution of the nuclei can also be created by hyperpolarization NMR techniques for which the spin population differences is increased by several orders of magnitudes compared with the thermal equilibrium conditions. This significantly increases the overall polarization of the nuclei thereby amplifying the magnetic resonance signal intensity.

The enhancement of the hyperpolarized magnetic resonance signal is limited by the relatively fast decay of the hyperpolarization due to spin-lattice relaxation (termed as T₁ relaxation time). This decay, combined with the initial level of the hyperpolarized signal, determines the temporal window of ability to detect the hyperpolarized nuclei. Known techniques of enriching the proton positions with deuterium were shown to prolong the T₁ relaxation times of carbon-13 in various compounds in a manner that is dependent on the compound's conformation in solution. The prolongation of T₁ values is attributed to a decrease in dipolar interaction that a particular nucleus experiences. However, because the dipolar interaction is only one of several relaxation mechanisms that affect the overall T₁ relaxation time, it is not possible to predict the extent of this effect for a particular nucleus in specific molecule within a specific medium (for example in the blood). Moreover, prolongation of T₁ in itself at times does not allow for practical and effective in vivo magnetic resonance detection of a compound or its metabolic fate when administered to a subject, since the sensitivity of detection is limited due to the low natural abundance of ¹³C nuclei, thereby yielding signals which are below the threshold of detection.

Most spin hyperpolarized MRI studies carried to date and specifically those involving dissolution DNP approach have been focused on metabolic imaging and thereby involved spectroscopic imaging, and the use of a compound that showed a chemical shift difference between its substrate form to its metabolic product.

The inventors of the present invention have shown that choline's carbon-13 demonstrates a long spin-lattice relaxation time (T₁) when directly bonded to deuterium atoms (WO/2011/024156). This was a prerequisite for utilization of choline as an agent for hyperpolarized magnetic resonance. All of the carbon positions in the choline moiety showed a long T₁ (≧28 s) upon substitution of directly bonded hydrogen atoms by deuterium atoms. The prolongation factor for the T₁ of the methylenic as well as the methyl positions ranged between 6 to 8 for the deuterated positions, i.e. deuterated carbon-13 nuclei in this moiety showed a T₁ that was longer 6-8 fold compared to the native protonated choline ion moiety.

There is still a need in the field of the invention to provide non-radioactive non-toxic compounds capable of providing a clear, quick, and safe diagnostic tool for different states, conditions, and disorders using magnetic resonance imaging.

### SUMMARY OF THE INVENTION

Since CDP-Choline has a much lower toxicity than that of choline, with LD₀ for intravenous administration being about 40 fold lower for CDP-Choline compared to choline and (Agut et al. 1983, Arzneimittel-Forschung), this compound provides a safer spectroscopic agent capable of being administered in large doses thus achieving a high and more accurate image for diagnosis.

In a first aspect the invention provides CDP-Choline comprising at least one isotopically labeled carbon atom directly bonded to at least one deuterium atom (commonly marked as D or ²H).

CDP-Choline (also named Citicoline) as used herein refers to the compound cytidine diphosphate-choline, (cytidine 5′-diphosphocholine and 5′-O-[hydroxy({hydroxy[2-(trimethylammonio)ethoxy]phosphoryl}oxy)phosphoryl]cytidine), having a choline moiety and a cytidine diphosphate moiety:

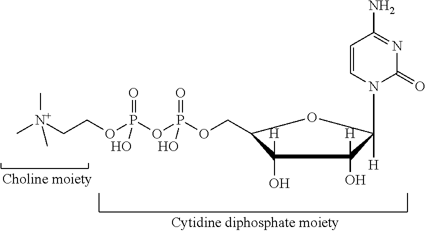

In some embodiments a CDP-Choline of the invention may also be derivatized by one or more fluorine atoms (i.e. fluorine atoms substituting a hydrogen atom of CDP-Choline).

In other embodiments, when referring to CDP-Choline it may also include any pharmaceutically acceptable salt thereof, thus including one or more suitable counter ion(s). CDP-Choline comprises positively charged quaternary nitrogen atom at the choline moiety. In other embodiments CDP-Choline may also include a further positively charged atom and/or a negative charged atom (for example a negatively charged oxygen at the cytidine diphosphate moiety).

The term “pharmaceutically acceptable salt”, as used herein, means those salts of CDP-Choline of the invention that are safe and effective for use in mammals and that possess the desired biological activity. Pharmaceutically acceptable salts include salts of acidic or basic groups present in CDP-Choline of the invention. Pharmaceutically acceptable acid addition salts include, but are not limited to, hydrochloride, hydrobromide, hydroiodide, nitrate, sulfate, bisulfate, phosphate, acid phosphate, iso nicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Pharmaceutically acceptable salts may also be formed with various amino acids. Suitable base salts include, but are not limited to, aluminum, calcium, lithium, magnesium, potassium, sodium, zinc, and diethanolamine salts. For a review on pharmaceutically acceptable salts see BERGE ET AL., 66 J. PHARM. SCI. 1-19 (1977), incorporated herein by reference.

The term “isotopically labeled atom” is meant to encompass an atom in a compound of the invention for which at least one of its nuclei has an atomic mass which is different than the atomic mass of the prevalent naturally abundant isotope of the same atom. Due to different number of neutrons in the nuclei, the atomic mass of isotopically labeled atoms is different. The total number of neutrons and protons in the nucleus represents its isotopic number. As will be appreciated by the description below, the isotopic labeling of specific atoms in a compound of the invention is achieved by techniques known to a person skilled in the art of the invention, such as for example synthesizing compounds of the invention from isotopically labeled reactants or isotopically enriching specific nuclei of a CDP-Choline a or derivative thereof.

When referring to a CDP-Choline or derivative thereof comprising at least one isotopically labeled atom, it should be understood to encompass a compound having said isotopically labeled atoms above the natural abundance of said at least one isotopically labeled atom. Thus, when said isotopically labeled atom is deuterium, said isotopical enrichment of said deuterium in a specific position in a compound of the invention, may be between about 0.015% to about 99.9%. When said isotopically labeled atom is ¹³C, said isotopical enrichment of said carbon in a specific position in a compound of the invention, may be between about 1.1% to about 99.9%. In some other embodiments when said isotopically labeled atom is ¹⁸F, said isotopical enrichment of said fluorine in a specific position in a compound of the invention, may be in between about 0.001% to about 100%. Thus, a compound or a composition of the invention may have different degrees of enrichment of isotopically labeled atoms.

When referring to said at least one isotopically labeled carbon atom being directly bonded to at least one deuterium atom it should be understood to encompass said isotopically labeled carbon atom having a hybridization of sp³ or sp², thus being able to connect to at least one deuterium atom via at least one sigma bond. In some embodiments said at least one isotopically labeled carbon atom may be directly bonded to more than one deuterium atom. In other embodiments said at least one isotopically labeled carbon atom is connected to 1, 2 or 3 deuterium atoms. In other embodiments said at least one isotopically labeled carbon atom may be further directly connected to at least one other isotopically labeled atom.

In some embodiments a CDP-Choline of the invention comprises at least two isotopically labeled carbon atoms, wherein at least one isotopically labeled carbon atom is directly bonded to at least one deuterium atom. In some embodiments said at least two isotopically labeled carbon atoms are directly bonded to one another (via a sigma or pi bond). In other embodiments, said at least two isotopically labeled carbon atoms are separated from each other by at least one atom (other than hydrogen or deuterium).

In further embodiments a CDP-Choline of the invention comprises one to five (i.e. 1, 2, 3, 4, or 5) isotopically labeled carbon atoms, wherein at least one isotopically labeled carbon atom is directly bonded to at least one deuterium atom. In some embodiments at least two of said at least one to five isotopically labeled carbon atoms are directly bonded to one another (via a sigma or pi bond). In other embodiments, at least two of said at least one to five isotopically labeled carbon atoms are separated from each other by at least one atom (other than hydrogen or deuterium).

In other embodiments each isotopically labeled carbon atom is directly bonded to at least one deuterium atom.

In further embodiments said at least one isotopically labeled carbon atom is a carbon of the choline moiety (i.e. the carbon atoms of the moiety (CH₃)₃N⁺CH₂CH₂—). Thus in some embodiments a moiety is isotopically labeled in accordance with the following non-limiting list: (CH₃)₃N⁺¹³CDHCH₂—, (CH₃)₃N⁺¹³CD₂CH₂—, (CH₃)₃N⁺CH₂¹³CDH—, (CH₃)₃N⁺CH₂¹³CD₂-, (CH₃)₃N⁺¹³CH₂¹³CDH—, (CH₃)₃N⁺¹³CDH¹³CDH—, (CH₃)₃N⁺¹³CD₂¹³CD₂-, ¹³CH₂D(CH₃)₂N⁺CH₂CH₂—, ¹³CHD₂(CH₃)₂N⁺CH₂CH₂—, ¹³CD₃(CH₃)₂N⁺CH₂CH₂—, (¹³CD₃)₂(CH₃)N⁺CH₂CH₂—, (¹³CD₃)₃N⁺CH₂CH₂—, (¹³CD₃)₃N⁺¹³CH₂CH₂—, (¹³CD₃)₃N⁺C¹³DHCH₂—, (¹³CD₃)₃N⁺¹³CD₂CH₂—, (¹³CD₃)₃N⁺¹³CH₂¹³CH₂—, (¹³CD₃)₃N⁺¹³CH₂¹³CDH—, (C¹³D₃)₃N⁺¹³CH₂¹³CD₂-, (C¹³D₃)₃N⁺¹³CD₂¹³CD₂-, (CH₃)₃N⁺¹³CD₂CD₂-, (CH₃)₃N⁺CD₂¹³CD₂-.

In other embodiments said isotopically labeled carbon atom is ¹³C (having 7 neutrons and 6 protons in carbon nucleus).

In other embodiments a CDP-Choline of the invention further comprises at least one isotopically labeled hydrogen atom. In some embodiments said at least one further isotopically labeled hydrogen atom is bonded to at least one isotopically labeled carbon atom. In further embodiments said at least one further isotopically labeled hydrogen atom is bonded to an atom adjacent to at least one isotopically labeled carbon atom.

In further embodiments a CDP-Choline of the invention further comprises at least one (additional) isotopically labeled carbon atom. In some embodiments said at least one further isotopically labeled carbon atom is directly bonded to said at least one isotopically labeled carbon atom. In other embodiments said at least one additional isotopically labeled carbon atom is adjacent to said at least one isotopically labeled carbon atom.

In other embodiments a CDP-Choline of the invention further comprises at least one isotopically labeled nitrogen atom (¹⁵N). In some embodiments said at least one isotopically labeled nitrogen atom is directly bonded to said at least one isotopically labeled carbon atom. In other embodiments said isotopically labeled nitrogen atom is adjacent to said at least one isotopically labeled carbon atom.

In other embodiments a CDP-Choline of the invention further comprises at least one radioactive fluorine atom (¹⁸F having 9 neutrons and 9 protons in fluorine nucleus). In some embodiments said at least one radioactive fluorine atom is directly bonded to said at least one isotopically labeled carbon atom. In other embodiments said radioactive fluorine atom is adjacent to said at least one isotopically labeled carbon atom.

In further embodiments a CDP-Choline of the invention, further comprising at least one radioactive carbon atom (¹¹C). In some embodiments said at least one radioactive carbon atom is directly bonded to said at least one isotopically labeled carbon atom. In other embodiments said radioactive carbon atom is adjacent to said at least one isotopically labeled carbon atom.

In further embodiments a CDP-Choline according to the invention is selected from the following list:
[1,1,2,2-D₄,2-¹³C]citicoline (FIG. 1)
[2,2-D₂,2-¹³C]citicoline
[2-D, 2-¹³C]citicoline
[1,1,2,2-D₄,1-¹³C]citicoline (FIG. 2)
[1,1-D₂,1-¹³C]citicoline
[1-D,1-¹³C]citicoline
[1,1,2,2-D₄,1,2-¹³C₂]citicoline
[1,2,3-D₁₃,1-¹³C]citicoline
[1,2,3-D₁₃,2-¹³C]citicoline
[1,2,3-D₁₃,1,2,3-¹³C₅]citicoline (FIG. 3)
[trimethylamine-¹⁵N]citicoline
[trimethylamine-¹⁵N,D₉]citicoline

As noted above in order to acquire an NMR signal of a particular nucleus of a compound there has to be a significant difference between the spin population energy levels of said nucleus. The strength of the NMR signal is linearly dependent on the number of nuclei at the low energy level. The difference between the population of a nucleus at high and low nuclear energy levels is the “polarization” of the nuclei, which is defined as P=CB₀/T, where C is a nucleus specific constant, B₀ is the magnetic field strength, and T is the absolute temperature. Under thermal equilibrium conditions, the polarization is relatively low thereby resulting in a very weak signal under standard clinical MRI scanners (at body temperature of about 37° C. for a magnetic field of 1.5 T, P (for ¹H) is approximately 5×10⁻⁶ and P (for ¹³C) is approximately 1×10⁻⁶).

In order to increase the polarization of a specific nucleus in a compound consequently creating an artificial, non-equilibrium distribution of the spin population of a nucleus, i.e. a “hyperpolarized” state, where the spin population difference is increased by several orders of magnitudes compared with the thermal equilibrium.

Thus, in some other embodiments a CDP-Choline according to the invention is in a hyperpolarized state. In some embodiments said hyperpolarization is achieved using dynamic nuclear polarization technique or para-hydrogen induced polarization.

Ex vivo hyperpolarization is achieved by means of dynamic nuclear polarization (DNP) techniques, such as the Overhauser effect, in combination with a suitable free radical (e.g. TEMPO, trityl radical and their derivatives). Hyperpolarization may also be performed ex-vivo using the Para-hydrogen Induced Polarization technique, and ortho-deuterium induced polarization. Ex-vivo hyperpolarization may also be performed by interaction with a metal complex and reversible interaction with para-hydrogen without hydrogenation of the organic molecule. These techniques have been described in U.S. Pat. No. 6,466,814, U.S. Pat. No. 6,574,495, and U.S. Pat. No. 6,574,496, and in Adams R. W. et al. (Science, 323, 1708-1711, 2009), the contents of which are incorporated herein by reference.

Ex vivo hyperpolarization of a compound of the invention is performed in order to reach a level of polarization sufficient to allow a diagnostically effective contrast enhancement of said agent. In some embodiments, said level of hyperpolarization may be at least about a factor of 2 above the thermal equilibrium polarization level at the magnetic field strength at which the MRI is performed. In some embodiments, said level of hyperpolarization is at least about a factor of 10 above the thermal equilibrium polarization level at the magnetic field strength at which the MRI is performed. In other embodiments, said level of hyperpolarization is at least about a factor of 100 above the thermal equilibrium polarization level at the magnetic field strength at which the MRI is performed. In yet further embodiments, said level of hyperpolarization is a factor of at least about 1000 above the thermal equilibrium polarization level at the magnetic field strength at which the MRI is performed. In other embodiments said level of hyperpolarization is a factor of at least about 10000 above the thermal equilibrium polarization level at the magnetic field strength at which the MRI is performed. In further embodiments said level of hyperpolarization is a factor of at least 100000 above the thermal equilibrium polarization level at the magnetic field strength at which the MRI is performed.

A hyperpolarized CDP-Choline or derivative thereof according to the invention comprises nuclei capable of emitting magnetic resonance signals in a magnetic field (e.g. nuclei such as ¹³C and ¹⁵N) and capable of exhibiting T₁ relaxation times between about 5 to about 70 sec (at standard MRI conditions such as for example at a field strength of 0.01-5T and a temperature in the range 20-40° C.). In some embodiments, said hyperpolarized CDP-Choline or derivative thereof according to the invention has T₂ relaxation times of ¹³C nucleus of between about 10 to about 10,000 msec.

In a further aspect the invention provides a composition comprising at least one CDP-Choline of the invention.

It is noted that a composition of the invention may comprise, in some embodiment, at least one CDP-Choline or derivative thereof according to the invention in a mixture with pharmaceutically acceptable auxiliaries, and optionally other therapeutic agents. The auxiliaries must be “acceptable” in the sense of being compatible with the other ingredients of the composition and not deleterious to the recipients thereof.

Compositions administrable to a subject include those suitable for oral, rectal, nasal, topical (including transdermal, buccal, and sublingual), vaginal or parenteral (including subcutaneous, intramuscular, intravenous, and intradermal) administration or administration via an implant. The compositions may be prepared by any method well known in the art of pharmacy. Such methods include the step of bringing in association a CDP-Choline or derivative thereof the invention with any auxiliary agent. The auxiliary agent(s), also named accessory ingredient(s), include those conventional in the art, such as carriers, fillers, binders, diluents, disintegrants, lubricants, colorants, flavoring agents, anti-oxidants, and wetting agents.

Compositions suitable for oral administration may be presented as discrete dosage units such as pills, tablets, dragees or capsules, or as a powder or granules, or as a solution or suspension. The active ingredient may also be presented as a bolus or paste. The compositions can further be processed into a suppository or enema for rectal administration.

The invention further includes a composition, as hereinbefore described, in combination with packaging material, including instructions for the use of the composition for a use as hereinbefore described.

For parenteral administration, suitable compositions include aqueous and non-aqueous sterile injection. The compositions may be presented in unit-dose or multi-dose containers, for example sealed vials and ampoules, and may be stored in a freeze-dried (lyophilized) condition requiring only the addition of sterile liquid carrier, for example water, prior to use. For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulizers or insufflators.

The compounds of the invention may be administered in conjunction with other compounds, including, but not limited to physiological saline and buffers, D₂O, vitamin C, radical residues, effective (minute) amounts of Gd-chelates such as Gd-DTPA, Gd-DOTA, Gd-EDTA, effective (minute) amount of biocompatible DNP glassing agents such as ethanol and glycerol, and other hyperpolarized compounds such as pyruvate, glucose, and deoxyglucose.

In a further aspect the invention provides a CDP-Choline of the invention or any derivative thereof, for use as a medical imaging agent. In some embodiments, said imaging is an uptake or distribution imaging. In further embodiments, said imaging is spectroscopic imaging. In further embodiments, said spectroscopic imaging is metabolic imaging.

The term “medical imaging agent” is meant to encompass an agent (compound) that upon its in vivo administration to a subject is able to provide a visual image of at least a part of the body of said subject, or any tissue thereof. Said imaging agents are monitored using a variety of different modalities, such as radiography, computed tomography (CT), ultrasonography, magnetic resonance imaging (MRI), and radionuclide imaging. Such imaging is used for clinical purposes (medical procedures seeking to reveal, diagnose or examine disease) or medical science (including the study of normal anatomy and physiology).

When using MRI, the same agent can be used as a spectroscopic imaging agent and as an uptake or distribution imaging agent. This is dependent on the acquisition protocol applied. If the acquisition method applied quenches (disregards) the chemical shift information, therefore the imaging is said to be an uptake imaging approach (or flow) but may not be considered a spectroscopic imaging approach. Only when the chemical shift information is preserved, than the imaging method and the imaging agent can provide spectroscopic imaging, i.e. reveal information on the metabolism that took place (or the chemical reaction that the agent underwent). Most MRI sensitive agents can provide MRI images indicative of one or more properties such as uptake, flow, wash-in and wash-out of tissue. However, spectroscopic agents are unique in the sense that only specific positions of the image's molecule may provide spectroscopic information and other positions in the same molecule cannot. These unique positions are those in which the chemical shift is sufficiently different between the agent and the relevant metabolic product.

The term “uptake or distribution imaging agent” is meant to encompass an agent which provides only one type of signal for making an image or that only one type of signal is acquired, processed or used, regardless of the chemical evolutions of the agent in the blood and body. More than one type of information can be obtained by imaging of an uptake imaging agent, however information on the chemical evolutions of the agent is not obtained in this approach. In most cases the signal of an uptake imaging agent will reflect the spatial distribution of the agent after its administration to a subject, in a semi-quantitative or quantitative manner.

The term “spectroscopic imaging agent” is meant to encompass an agent that provides spectroscopic data related to the chemical evolution of the agent—specifically, in the case of metabolism—at least two different images or measurements can be obtained: one for the distribution of the agent and the other for the distribution of the metabolic product (or products) of the agent. After the spectroscopic imaging data are collected, processing of this data set produces an NMR spectrum in each volume element of the image. These spatially distributed spectra can then be processed to form metabolic images in a way that each volume element in the image reflects the intensity of a specific signal in the spectrum. For example, the intensity of the substrate signal in the spectra may vary in the image plane. This image of the substrate signal intensity spatial distribution can now be referred to as an image of the substrate, obtained from the spectroscopic imaging data. In the same manner, from the same parent spectroscopic imaging data an image of the product can be obtained. In this way, images of at least two distinct chemical entities (the substrate and the product) are produced.

CDP-Choline may be used as either an uptake/distribution imaging agent or spectroscopic imaging agent.

In some embodiments, for applications requiring metabolic information and differentiation between the agent and its metabolic products, CDP-Choline will be used as a spectroscopic imaging agent, using the stable-isotope labeling at the methylene positions which provide chemical shift differences between choline and its metabolites. Medical applications where CDP-Choline is used as a spectroscopic (metabolic) imaging agent include, but are not limited to: applications that require quantification of acetylcholine production rate in the brain (for example for the detection and treatment monitoring of Alzheimer's disease); applications that require quantification of phosphocholine production (for example for the characterization of tumors and evaluation of the aggressiveness, prognosis, and treatment response of malignant tumors).

Without wishing to be bound by theory it is noted that CDP-Choline is metabolized in vivo to choline and this metabolism activates the compound in the sense that choline is transported across cellular membranes and then undergoes metabolism in the cells. The cellular metabolism of choline (for example to acetylcholine, and/or phosphocholine, and/or betaine) provides a further diagnostic probe.

In other embodiments, for applications requiring imaging information without differentiation between the agent and its products, CDP-Choline will be used as an uptake/distribution imaging agent. Under these embodiments, all of the choline moiety carbon positions may be labeled with ¹³C and fully deuterated, to increase the SNR of the image and the T₁ of all positions. Alternatively, in other embodiments, in order to increase the T₁ of the agent further ¹⁵N-labeled or [¹⁵N,D₉]-labeled CDP-Choline may be used. Medical applications where CDP-Choline is used as an uptake/distribution imaging agent include, but are not limited to: applications that require quantification of choline uptake rate or observation of choline accumulation in the brain (for example for the detection and treatment monitoring of Alzheimer's disease); applications that require quantification of choline uptake rate or observation of choline accumulation (for example for the characterization of tumors and evaluation of the aggressiveness, prognosis, and treatment response of malignant tumors). The term “choline accumulation” is meant to encompass the accumulation of choline and any of its metabolic products in specific area or tissues. In a further aspect the invention provides a CDP-Choline of the invention or any derivative thereof, for use in imaging of at least one body part and/or tissue.

The term “imaging of at least one body part and/or tissue” is meant to encompass the image produced upon monitoring the uptake, distribution and/or metabolism of a spectroscopic agent visualizing detailed internal structures of at least one body part and/or a tissue thereof by magnetic resonance techniques. Such techniques provide good contrast between the different soft tissues of the body, which make them especially useful in imaging the brain, muscles, the heart, and medical conditions such as cancer compared with other medical imaging techniques such as computed tomography (CT) or X-rays. Unlike CT scans or traditional X-rays, magnetic resonance does not use ionizing radiation.

In another one of its aspects the invention provides a CDP-Choline of the invention or any derivative thereof, for use in diagnosing and evaluating a state, condition, or disease.

The term “diagnosing and evaluating a state, condition, or disease” is meant to encompass any process of investigating, identifying, recognizing and assessing a state, condition, disease, or disorder of the mammalian body (including its brain). A diagnosis according to the present invention using a CDP-Choline or derivative thereof according to the invention includes, but is not limited to the objective quantitative diagnosis of a condition or disease, prognosis of a condition or disease, genetic predisposition of a subject to have a condition or disease, efficacy of treatment of a therapeutic agent administered to a subject (either continually or intermittently), quantification of neuronal function, diagnosis and evaluation of a psychiatric, neurodegenerative, and neurochemical diseases and disorders, affirmation of a therapeutic agent activity, determination of drug efficacy, characterization of masses, tumors, cysts, blood vessel abnormalities, and internal organ function; quantification of brain, kidney, liver, and other organs' function; evaluation and determination of the level of anesthesia, comatose states, and the brain regions affected by stroke or trauma and their penumbra, kidney, liver, and muscle function, examination of the action, response or progress of therapy (involving medicinal and non-medicinal treatment) aimed at alleviating or curing psychiatric and neurodegenerative diseases and disorders. Said diagnosis and/or evaluation is achieved by administration to a subject a hyperpolarized CDP-Choline compound of the invention, or a composition comprising a hyperpolarized CDP-Choline of the invention and monitoring its distribution and/or metabolites in said subject by means of magnetic resonance techniques. The output image provided by said magnetic resonance techniques is able to provide to a professional person means to determine, diagnose and evaluate a state, condition, or disease existing in said subject.

In some embodiments, said state, condition, or disease diagnosed and/or evaluated using a CDP-Choline of the invention is selected from:

Oncologic related states, diseases or conditions including but not limited to: Tumor staging and differentiation, tumor grading, determination of tumor penetration into surrounding tissue, monitoring response to treatment, distant metastases, systemic metastasis, lymph node staging, recurrent disease, cancer imaging, radiation oncology, central nervous system tumors and cancer, head and neck cancer, brain cancer, thyroid cancer and thyroid imaging, anaplastic carcinomas of thyroid, lung cancer, non-small cell lung cancer, lymphoma and myeloma, malignant melanoma, breast cancer, esophageal cancer, colorectal carcinoma, pancreatic and hepatobiliary cancer, gynecological tumors, cervical and uterine cancers, ovarian cancer, endometrial cancer, genitourinary malignancies, sarcomas, gastrointestinal stromal tumors, neuroendocrine tumors, gastrinoma, glomus tumor, liver metastasis, astrocytoma, pilocytic astrocytoma, glioblastoma, carcinoma of unknown primary including paraneoplastic neurological syndromes, carcinoid tumor, cancer in pediatric patients, gallbladder carcinoma, hypoxia imaging, angiogenesis imaging, antiangiogenic therapeutic strategies, lymph node metastasis, Breslow's depth and thickness determination, bone lesions, bladder cancer, brown fat and hibernoma, cholangiocarcinomas, pulmonary node detection, ganglioglioma, gliomatosis cerebri, malignant degeneration of low grade glioma, prostate cancer, renal cancer, testicular cancer, genitourinary tract cancer, kidney cancer, hepatobiliary tumors, benign tumors—adrenal adenoma, and adrenal hypertrophy;

Neurologic related states, diseases or conditions including but not limited to: movement disorders, stroke, epilepsy, epilepsy in childhood, extratemporal lobe epilepsy, dementia, amphetamine induced activity, Alzheimer's disease, early onset familial Alzheimer's disease, cerebral amyloid angiopathy, dementia with Lewy bodies, frontotemporal lobal degeneration, mild cognitive impairment, Parkinson's disease, atypical parkinsonian disorders, brain development, central nervous system tumors, cerebral blood flow, interictal imaging, ictal imaging, infantile spasms, Lennox-Gastaut syndrome, normal aging imaging, cerebral oxygen metabolism, stroke, corticobasal degeneration, frontal hypometabolism, and Gilles de la Tourette syndrome;

Psychiatric related states, diseases or conditions including but not limited to: affective disorders, bipolar disorder, depression, major depressive disorder, alcohol abuse, substance abuse, cocaine abuse, anxiety disorders, personality disorders, schizophrenia, schizoaffective disorder, social fobia, post-traumatic stress disorder, and obsessive compulsive disorder;

Cardiac and vascular related states, diseases or conditions including but not limited to: evaluation of myocardial perfusion, myocardial viability, oxidative metabolism and cardiac efficiency, hypertension, myocardial neurotransmitter imaging, absolute myocardial blood flow assessment, congestive heart failure, aortic graft, arterial plasma measurement, atheroschlerosis, blood vessel formation, cardial resynchronization assessment, coronary artery disease assessment, coronary viability assessment, myocardial involvement in endocrine disorders, cardiac stem cell therapy, cardiomyopathy, pediatric cardiology, dilated cardiomyopathy, myocardial reserve assessment, dobutamine stress test, heart innervations, heart transplantation, valvular heart disease, ischemic myocardium, imaging the neovasculature, imaging of blood volume and vascular permeability;

Infection and inflammation related states, diseases or conditions including but not limited to: infection in pediatric patients, cardiorespiratory infectious processes, fever of unknown origin, focal soft tissue infections, foreign body inflammatory reaction, infection and inflammation in immune compromised patients, infection superimposed on malignancy, inflammation in children, inflammatory bowel disease (IBD), colitis, Crohn's disease, musculoskeletal inflammatory process, inflammatory joint disease, joint prosthesis infection, metallic implant infection, osteomyelitis, sarcoidosis, vascular infection, vascular graft infection, vasculitis, vulnerable atherosclerotic plaque, rheumatoid arthritis, systemic and local autoimmune diseases, AIDS infection, differentiating inflammation from malignancy, pyogenic infection, parasitic, viral infection, and bacterial infection;

Kidneys related states, diseases or conditions including but not limited to: Alport syndrome, renography, captopril renography, renal artery stenosis, and kidney transplantation;

General states, diseases or conditions including but not limited to: mapping and/or monitoring over time of abnormal metabolism, mapping of metabolic response to extrinsic or intrinsic modulation, angiography, catheter angiography, interventional radiology, neuro-interventional radiology, hemorrhagic infarction, head injuries, brain trauma conditions, and hemorrhagic stroke.

In some embodiments a state, condition, or disease is selected from cancer (such as for example breast cancer, head and neck cancer, brain cancer, urological cancer, gynecological cancer), neurological condition or disease, neurodegenerative disease, psychiatric condition or disease, cardiovascular condition or disease, vascular condition or disease, inflammatory condition or disease.

In another one of its aspects the invention provides a use of CDP-Choline of the invention, for the manufacture of a spectroscopic imaging agent. In some embodiments said agent is formulated as a composition for administration in diagnosing a condition, disease or disorder as will be detailed herein below.

In a further aspect the invention provides a use of CDP-Choline of the invention for the manufacture of a composition for diagnosing and evaluating a condition or disease.

In some embodiments, said composition comprises said CDP-Choline of the invention in an amount of between about 0.1 mg/Kg to about 500 mg/Kg. In some embodiments, said composition comprises said CDP-Choline of the invention in an amount of between about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 30, 40, 50, 60, 70, 80, 90, 100, 150, 200, 250, 300, 350, 400, 450, 500 mg/Kg.

In a further aspect the invention provides a method for diagnosing and evaluating a state, condition, or disease in a subject, said method comprising:
hyperpolarizing at least one CDP-Choline according to the invention or a composition thereof;
administering to said subject an effective amount of at least one hyperpolarized CDP-Choline;
imaging of said hyperpolarized at least one CDP-Choline;
thereby diagnosing said state, condition, or disease.

The term “monitoring” as used herein is meant to encompass the quantitative and/or qualitative detection and observation of a hyperpolarized CDP-Choline or derivative thereof according to the invention administered to said subject. Monitoring may be performed by any non-invasive or invasive imaging method, including, but not-limited to magnetic resonance spectroscopy, magnetic resonance imaging, magnetic resonance spectroscopic imaging, and PET.

In some embodiments said imaging is performed by means of magnetic resonance imaging.

In other embodiments, said magnetic resonance spectroscopy is performed using a double tuned ¹³C/D RF coil or a triple tuned ¹³C/D/H RF coil. Due to possible coupling between deuterium nuclei and ¹³C-nucleus, and/or protons, the signals ¹³C-signals are split, their intensity is diminished and the signal width is broadened. In order to allow visibility of the agent's or its metabolite's signals it is sometimes necessary to improve on the line-width of this signal and increase its intensity. This may be achieved by using a double tuned ¹³C/²H RF coil or a triple tuned ¹³C/D/H RF coil or another combination of such coils that is capable of performing deuterium decoupling and/or proton decoupling during the ¹³C acquisition. Various coil design possibilities such as a saddle coil, a birdcage coil, a surface coil, or combinations thereof are suitable for this purpose.

In further embodiments, said effective amount of hyperpolarized at least one CDP-Choline is between about 0.1 mg/Kg to about 500 mg/Kg.

In some other embodiments, said subject is administered with consecutive doses of said hyperpolarized CDP-Choline. Under these embodiments, the resulting images or spectra are optionally combined.

In further embodiments, said diagnosis and evaluation is performed during or after said subject is administered with at least one therapeutic or modulating agent.

In some embodiments, said imaging is a metabolic imaging or non-metabolic imaging of said CDP-Choline. In other embodiments, said imaging is a spatial distribution imaging of said at least one CDP-Choline. In other embodiments, said imaging is an uptake imaging of said at least one CDP-Choline.

In another aspect the invention provides a kit or system comprising at least one component containing at least one CDP-Choline comprising at least one isotopically labeled carbon atom directly bonded to at least one deuterium atom, means for administering said at least one CDP-Choline and instructions for use.

In some embodiments said kit or system further comprising at least one imaging agent, optionally in a separate component.

In further embodiments said kit or system of the invention is used in imaging at least one body part or tissue.

In other embodiments said kit or system of the invention is used in diagnosing and evaluating a state, condition, or disease.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 shows the chemical structure and labeled positions of [1,1,2,2-D₄, 2-¹³C]citicoline.

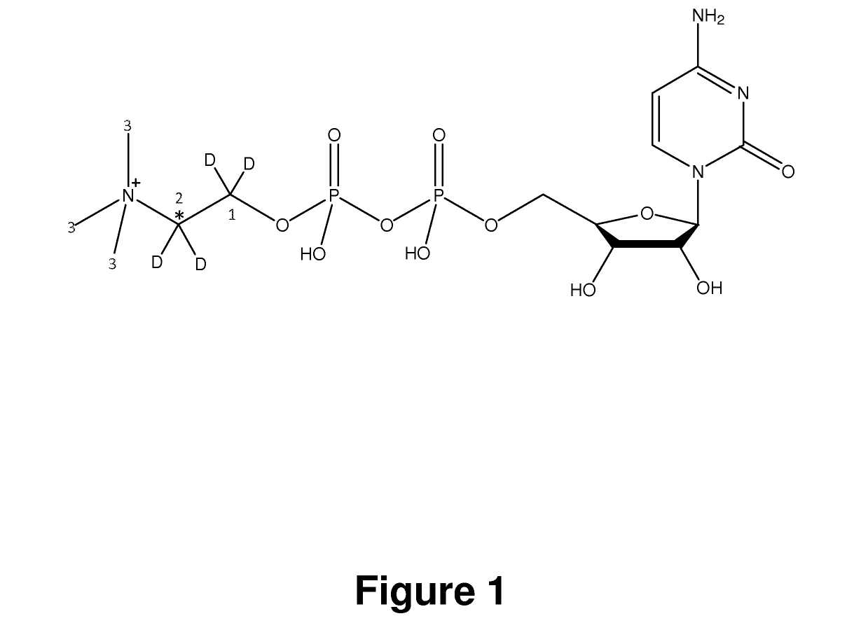

FIG. 2 shows the chemical structure and labeled positions of [1,1,2,2-D₄, 1-¹³C]citicoline.

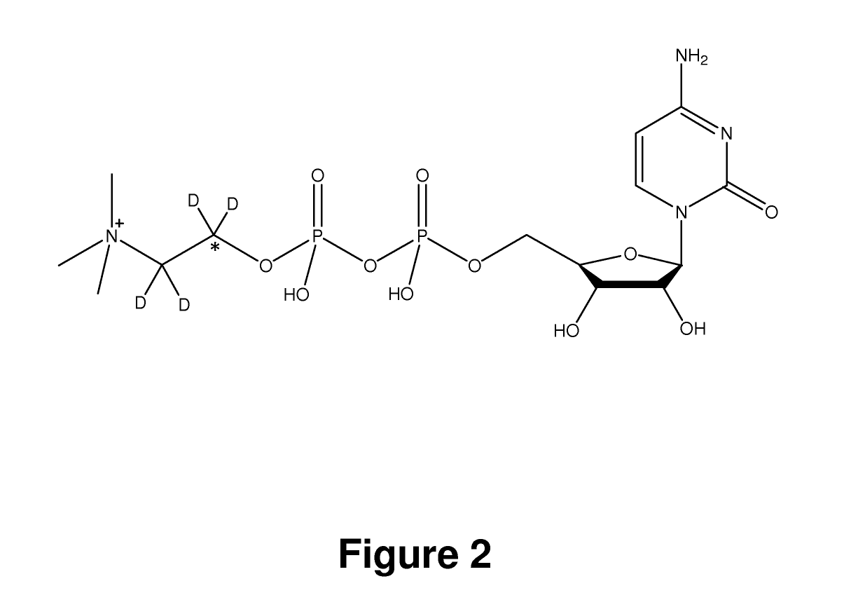

FIG. 3 shows the chemical structure and labeled positions in [1,2,3-D₁₃, 1,2,3-¹³C₅]citicoline.

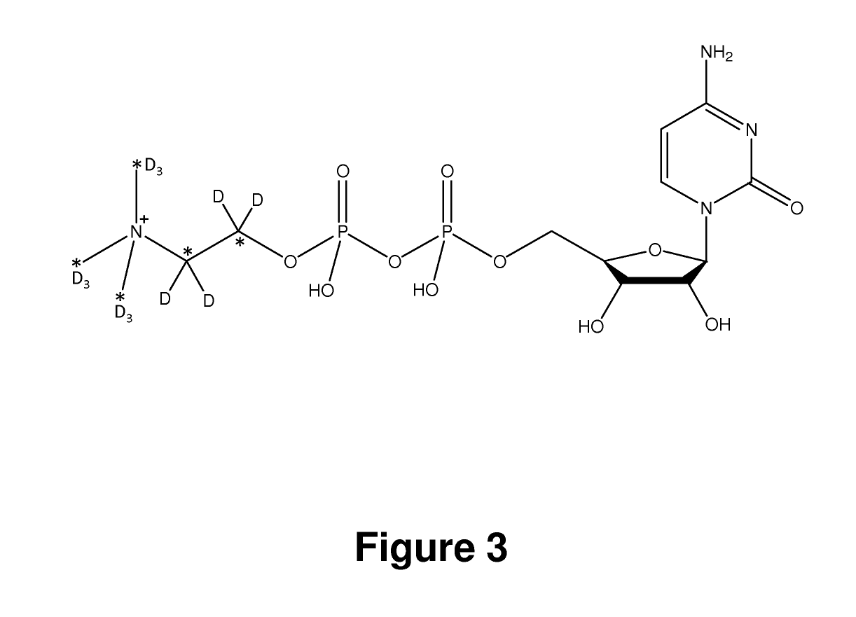

FIG. 4 shows the chemical structure of CDP-choline marking the nuclei types.

FIG. 5 shows the ³¹P-NMR spectra of CDP-Choline in water.

FIG. 6 shows the region of P1 of CDP-Choline in the ³¹P-NMR of CDP-Choline in water. The spectrum has been processed with apodization (SinSquare 40). The enhanced resolution reveals the multiplicity of the peak.

### DETAILED DESCRIPTION OF EMBODIMENTS

CDP-Choline can be synthesized from choline phosphate and cytidine triphosphate (CTP) via the enzymatic reaction of choline-phosphate cytidylyltransferase (EC 2.7.7.15). This is an enzyme that catalyzes the following chemical reaction:

CTP+choline phosphate [symbol] diphosphate+CDP-Choline

Another synthetic route, although with a lower yield may be carried out by reacting the two compounds at 70° C. for 24 h (Mar et al. 1987 Origins of Life “Non-enzymatic synthesis of the coenzymes, uridine diphosphate glucose and cytidine diphosphate choline, and other phosphorylated metabolic intermediates”).

CDP-Choline sodium salt dihydrate was obtained from the Sigma-Aldrich Co. (Israel) and was dissolved in distilled water to a concentration of 180 mM as a storage solution due to its hygroscopic properties.

Two Samples of CDP-Choline were Prepared:

1) CDP-Choline in Water

3.3 mg CDP-Choline were dissolved in 700 μL distilled water containing 10% deuterated water (85.7 mM solution) and stored in a 5 mm NMR tube in the freezer until assay.

2) CDP-Choline in Plasma

Two units of expired plasma (about 250 cc per unit) from blood type AB and O were obtained frozen from the blood bank section at Hadassah Medical Center. Plasma was defrosted in the refrigerator overnight. 565 μl of plasma solution was mixed with 75 IA distilled water and transferred to a 5 mm NMR tube. The solution was kept on ice until NMR analysis the same day. 60 μl CDP-Choline was added to the solution and ³¹P-NMR was recorded 37 s from the time CDP-Choline mixed with the plasma solutions in the NMR tube. The Ionic strength of the solution was 315.04 mM and the concentration of CDP-Choline 15.4 mM.

Spectroscopy Experiment

NMR Spectrometers

Spectra were obtained on an 11.8T high-resolution spectrometer (Varian, Palo Alto, Calif., USA) with a 5 mm direct multi-nuclei probe.

³¹P-NMR

Spectral parameters for CDP-Choline in water were: 37° C., 90 degree pulse, acquisition time of 1.6 s, relaxation delay of 1.0 s, 8 transients. The unmodified free induction decay was directly Fourier transformed. Spectra were recorded continuously starting at 37 sec from CDP-Choline addition to the plasmafor 10 minutes. Then, spectra were acquired every 20 min for one hour.

T₁ Experiment

T₁ measurements were performed for CDP-Choline in water and in plasma with the standard inversion recovery pulse sequence. The data were fitted to the standard inversion recovery equation using the MATLAB software (The MathWorks Inc., Natick, Mass., USA).

Results

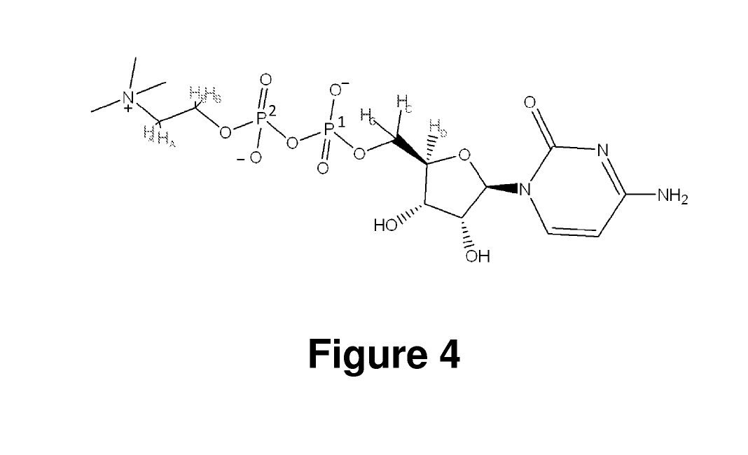

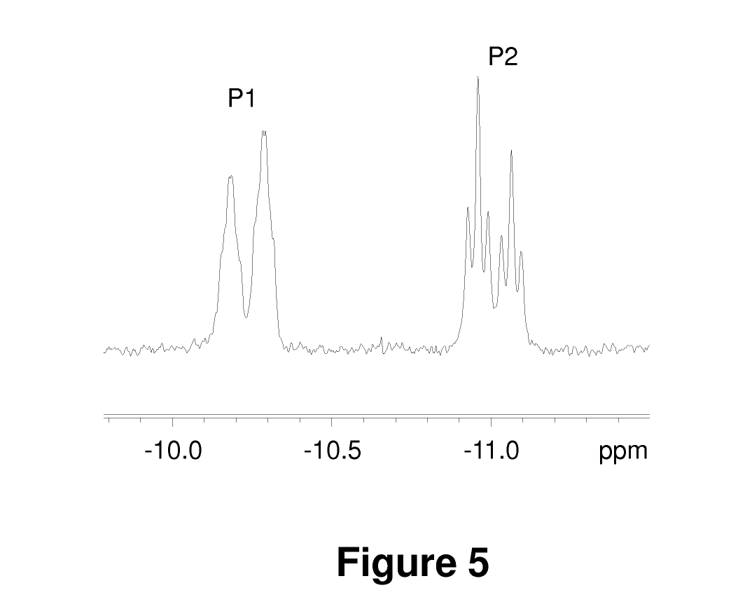

The various interacting nuclei types in CDP-choline are shown in FIG. 4. The ³¹P-NMR spectrum of CDP-Choline in water (85.7 mM) in FIG. 5 shows two signals: a doublet of triplet at −11.01 ppm and a doublet at −10.23 ppm. The doublet which appears at −10.23 ppm was assigned to the phosphate next to the nucleotide (P1), due to the effect of deshielding from the electron cloud surrounding the oxygen atoms and the ribose and cysteine rings. The signal at −11.01 ppm signal (P2) is a doublet of triplet due to additional coupling between the ³¹P nucleus and the two neighboring hydrogen nuclei in the choline molecule.

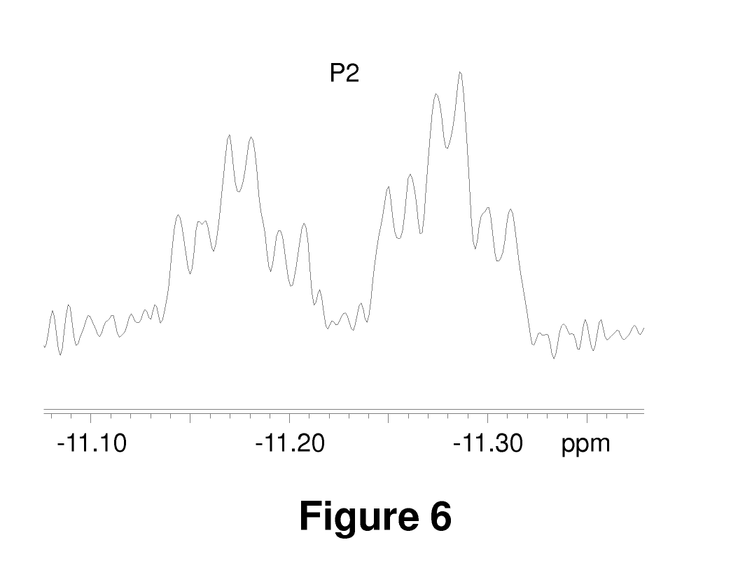

After resolution enhancement in the processing of the spectrum, each peak from the P1 signal is split into a triplet of doublet due to additional coupling with neighboring hydrogen in the nucleotide (see in FIG. 6). The signal is split into a triplet due to coupling with H_{C} while H_{D} splits each peak inside the triplet into a doublet. Apparently the coupling in between P2 and H_{A} was too small to be detected. The coupling constants are listed in Table 1. The spin-lattice relaxation time for the phosphate atoms are reported in Table 2 below.

TABLE 1
Homogenous and heterogeneous coupling
constant for CDP-Choline

| Coupling constant | Hz |
|---|---|
| J(P₁-P₂) | 21.5 |
| J(P₂-H_{B}) | 6.4 |
| J(P₁-H_{C}) | 5.2 |
| J(P₁-H_{D}) | 2.2 |

TABLE 2
T₁ values for the CDP-Choline phosphates

| T₁ (s) | H₂O | PLASMA |
|---|---|---|
| P₁ | 1.2 (0.9-1.5) | 2.0 (1.9-2.0)* |
| P2 | 1.4 (1.3-1.6) | 2.6 (2.4-2.8) |

*(confidence interval)

Surprisingly, the ³¹P spectrum of the CDP-choline sample in plasma was not modified throughout the measurement, indicating stability of the compound in plasma and most likely in whole blood as well. The fine spectral properties of the phosphorus spectrum indicated that indeed the entire content of phosphorus in the sample remained as CDP-choline and was not degraded throughout the measurement time, i.e. up to one hour.

Furthermore, it was unexpected to find that the relaxation time of the phosphorus nuclei is longer in plasma as compared to water. The T₁ of the carbon-13 nuclei in the choline moiety of CDP-choline was found to be about 2-3 s at 11.8 T. Deuteration of these positions is expected to result in a T₁ of 12-24 s, without shortening by the plasma environment.

1. CDP-Choline comprising at least one isotopically labeled carbon atom directly bonded to at least one deuterium atom.

2. CDP-Choline according to claim 1, comprising at least two isotopically labeled carbon atoms, wherein at least one isotopically labeled carbon atom is directly bonded to at least one deuterium atom.

3. (canceled)

4. (canceled)

5. CDP-Choline according to claim 1, wherein said carbon is a carbon of the choline moiety.

6. (canceled)

7. CDP-Choline according to claim 1, having T₁ relaxation time values of ¹³C nuclei of between about 5 to about 70 sec.

8. CDP-Choline according to claim 1, further comprising at least one of: at least one isotopically labeled hydrogen atom, at least one isotopically labeled carbon atom, at least one isotopically labeled nitrogen atom, at least one radioactive fluorine atom (18F), at least one radioactive carbon atom (11C).

9. (canceled)

10. (canceled)

11. (canceled)

12. (canceled)

13. CDP-Choline according to claim 1 selected from the following list:
[1,1,2,2-D₄,2-¹³C]citicoline
[2,2-D₂, 2-¹³C]citicoline
[2-D, 2-¹³C]citicoline
[1,1,2,2-D₄,1-¹³C]citicoline
[1,1-D₂,1-¹³C]citicoline
[1-D,1-¹³C]citicoline
[1,1,2,2-D₄,1,2-¹³C₂]citicoline
[1,2,3-D₁₃, 1-¹³C]citicoline
[1,2,3-D₁₃, 2-¹³C]citicoline
[1,2,3-D₁₃,1,2,3-¹³C₅]citicoline
[trimethylamine-¹⁵N]citicoline
[trimethylamine-¹⁵N,D₉]citicoline.

14. CDP-Choline according to claim 1, being in a hyperpolarized state.

15. (canceled)

16. A composition comprising at least one CDP-Choline comprising at least one isotopically labeled carbon atom directly bonded to at least one deuterium atom.

17. A composition according to claim 8, further comprising at least one imaging agent selected from: D₂O, Gd-chelates, vitamin C, stable free radicals.

18. (canceled)

19. (canceled)

20. (canceled)

21. (canceled)

22. (canceled)

23. (canceled)

24. (canceled)

25. A method for diagnosing and evaluating a state, condition, or disease in a subject, said method comprising:
hyperpolarizing at least one CDP-Choline according to claim 1 or a composition thereof;
administering to said subject an effective amount of at least one hyperpolarized CDP-Choline;
imaging said hyperpolarized at least one CDP-Choline;
thereby diagnosing said state, condition, or disease.

26. A method according to claim 25, wherein said monitoring is performed by means of magnetic resonance imaging.

27. A method according to claim 25, wherein said effective amount of hyperpolarized at least one CDP-Choline is between about 0.1 mg/Kg to about 500 mg/Kg.

28. A method according to claim 25, wherein said subject is administered with consecutive doses of said hyperpolarized CDP-Choline.

29. (canceled)

30. A method according to claim 25, wherein said diagnosis and evaluation is performed during or after said subject is administered with at least one therapeutic or modulating agent.

31. A method according to claim 25, wherein said imaging is a metabolic imaging of said at least one CDP-Choline.

32. A method according to claim 25, wherein said imaging is a spatial distribution imaging of said at least one CDP-Choline.

33. A method according to claim 25, wherein said imaging is an uptake imaging of said at least one CDP-Choline.

34. A method according to claim 25, wherein said state, condition or disease is selected from cancer, neurological condition or disease, neurodegenerative disease, psychiatric condition or disease, cardiovascular condition or disease, vascular condition or disease, inflammatory condition or disease.

35. A kit comprising at least one component containing at least one CDP-Choline comprising at least one isotopically labeled carbon atom directly bonded to at least one deuterium atom, means for administering said at least one CDP-Choline and instructions for use, in imaging at least one body part or tissue or diagnosing and evaluating a state, condition, or disease.

36. A kit according to claim 35, further comprising at least one imaging agent, optionally in a separate component.

37. (canceled)

38. (canceled)
